(19) United States
(12) Patent Application Publication
(10) Pub. No.: US 2017/0342106 A1
(43) Pub. Date: Nov. 30, 2017

(54) METHOD OF MAKING PRODRUGS AND TARGETED THERAPEUTIC COMPOUNDS

(71) Applicant: Inspyr Therapeutics, Inc., Westlake Village, CA (US)

(72) Inventors: John K. Lynch, Cedarburg, WI (US); Jeffrey Hutchison, Sheboygan, WI (US); Thomas R. Benson, Brookfield, WI (US)

(73) Assignee: Inspyr Therapeutics, Inc., Westlake Village, CA (US)

(21) Appl. No.: 15/527,637

(22) PCT Filed: Nov. 10, 2015
(86) PCT No.: PCT/US2015/060039

§ 371 Date: May 17, 2017

Related U.S. Application Data
(60) Provisional application No. 62/081,385, filed on Nov. 18, 2014.

(51) Int. Cl. C07K 5/02; A61K 47/65; G01N 33/574; C07K 7/06

(52) U.S. Cl. CPC C07K 5/0205; G01N 33/574; A61K 47/65; C07K 7/06

(57) ABSTRACT

Provided is a method for making the compound of Formula 1. Various compounds utilized in that method are also provided, as are methods of making those compounds. Also provided is a compound having the formula XO—CO—(CH₂)ₙNH₂, where n is an integer greater than 2. A method of making that compound is additionally provided. Further provided is a method of making a prodrug of a bioactive compound.

### CROSS-REFERENCE TO RELATED APPLICATIONS

This application claims the benefit of U.S. Provisional Application No. 62/081,385 filed Nov. 18, 2014, which is incorporated by reference herein in its entirety.

### FIELD OF THE INVENTION

The present application generally relates to methods for synthesizing organic compounds. More specifically, the invention relates to methods of making prodrug compounds, for example prodrugs useful against cancer, and in particular, to methods of making relatively non-toxic prodrugs that are cleaved by peptidases such as prostate specific membrane antigen (PSMA) to release a cytotoxic therapeutic compound, e.g., thapsigargin or other compounds (see, for example, U.S. Pat. No. 7,468,354), where, in some embodiments, the prodrug comprises the thapsigargin derivative 8-O-(12-aminododecanoyl)-8-O-debutanoyl thapsigargin (12ADT) linked to the aspartic acid of a peptide having the sequence Asp-Glu*Glu*Glu*Glu, wherein at least one of the bonds designated with * is a gamma carboxy linkage, and having the formula of Formula 1:

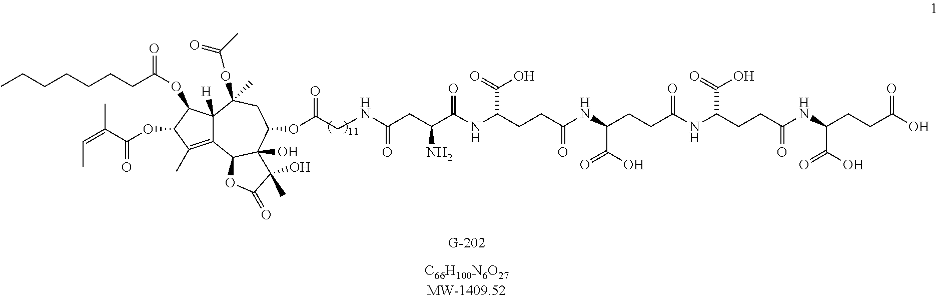

as well as methods of making certain intermediates thereof. The invention also relates to compounds and intermediates obtained by the processes herein set forth.

### BACKGROUND OF THE INVENTION

### Description of the Related Art

A peptide prodrug compound identified as G-202, and comprising the thapsigargin derivative 8-O-(12-aminododecanoyl)-8-O-debutanoyl thapsigargin (12ADT) linked to the aspartic acid of a peptide having the sequence Asp-Glu*Glu*Glu*Glu, wherein at least one of the bonds designated with * is a gamma carboxy linkage and having the structural formula of Formula 1:

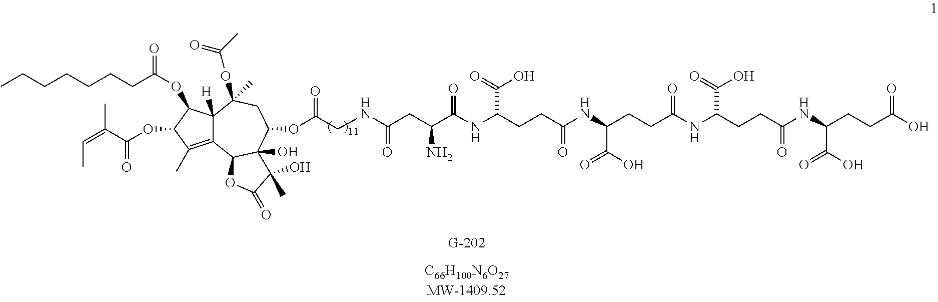

has been set forth and described in U.S. Pat. Nos. 7,767,648 and 7,468,354. Injectable cancer compositions comprising G-202 and methods and compositions for treating hepatocellular carcinoma using G-202 are also disclosed in U.S. Provisional Patent Application 61/714,662 and 61/693,273. Additionally, U.S. Provisional Patent Application 61/791,909 teaches various methods of making G-202 and other compounds.|

One major challenge for a process to produce G-202 is from the lack of crystallinity of any of the intermediates or final active pharmaceutical ingredient (API). This precludes the use of crystallization for removal of impurities at any point in the synthesis. This constraint makes it essential that the reactions be highly efficient and generate little to no impurities. In addition, the lack of crystallinity increases the value of alternate purification processes such as aqueous extractions, polar/non-polar organic partitioning, precipitation, trituration and efficient chromatographic purification. This process disclosed in U.S. Provisional Patent Application 61/791,909 provides an effective synthetic strategy to generate pure G-202. The present invention provides an alternative strategy for producing G-202 and other prodrugs, which provides certain advantages to the methods described in the above applications and patents.

### BRIEF SUMMARY OF THE INVENTION

Provided herewith is a method for making the compound of Formula 1:

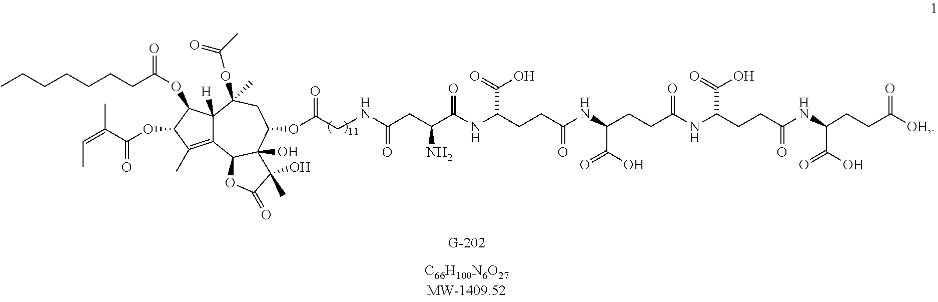

The method comprises

(a) reacting the compound of Formula 2:

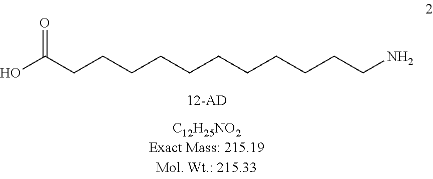 [SMILES: NCCCCCCCCCCCC(=O)O]

with X—OH in the presence of an acid catalyst to produce the compound of Formula 3a

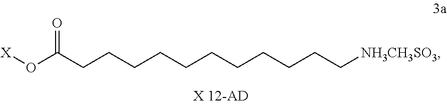

where X is a substituent that forms an ester that can be cleaved from the compound of Formula 5a

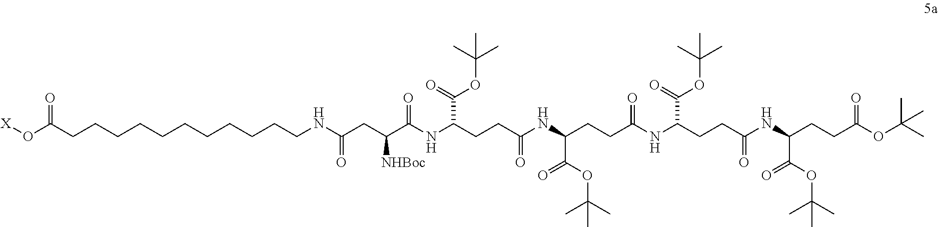

to produce the compound of Formula 6:

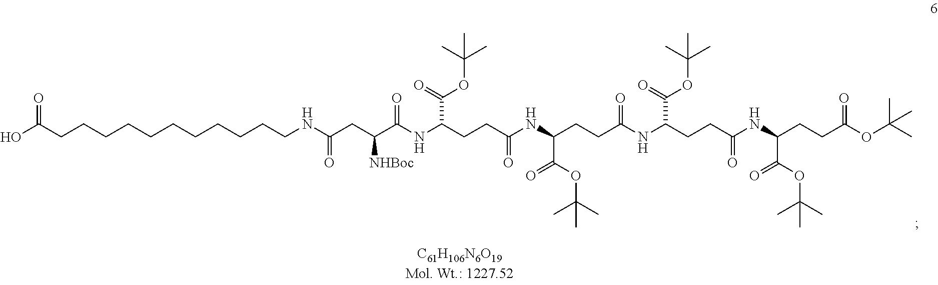 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]

(b) reacting the compound of Formula 3a with the compound of Formula 4:

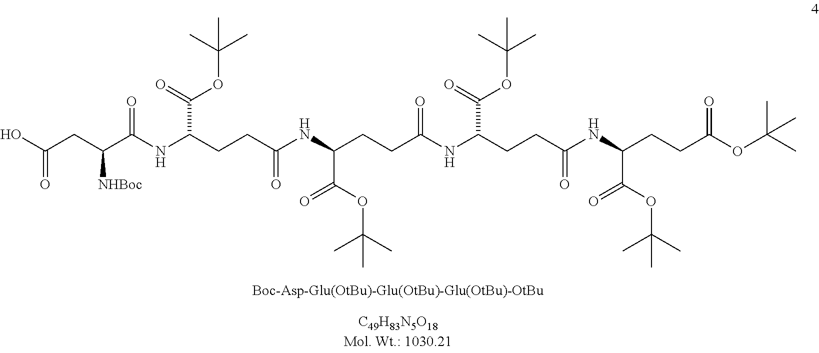 [SMILES: CC(C)(C)OC(=O)CC[C@H](NC(=O)CC[C@H](NC(=O)CC[C@H](NC(=O)CC[C@H](NC(=O)[C@H](CC(=O)O)NC(=O)OC(C)(C)C)C(=O)OC(C)(C)C)C(=O)OC(C)(C)C)C(=O)OC(C)(C)C)C(=O)OC(C)(C)C]

to produce the compound of Formula 5a;

(c) removing the X group from the compound of Formula 5a to produce the compound of Formula 6:

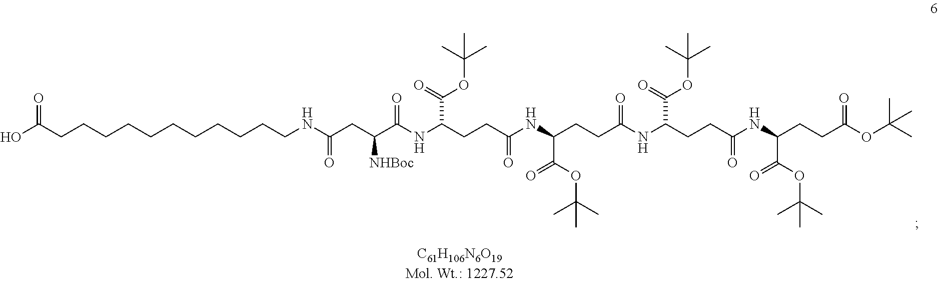 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]

(d) reacting the compound of Formula 6 with the compound of Formula 7:

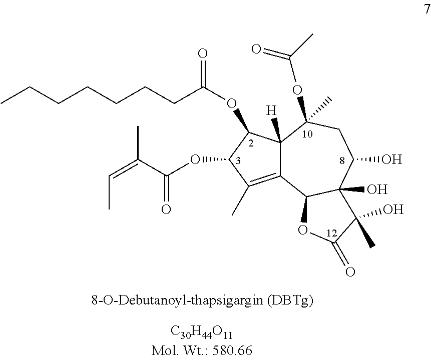 [SMILES: C/C=C(/C)C(=O)O[C@H]1C(C)=C2[C@H]([C@@H]1OC(=O)CCCCCCC)[C@@](C)(OC(C)=O)C[C@H](O)[C@@]1(O)[C@H]2OC(=O)[C@@]1(C)O]

to produce the compound of Formula 8:

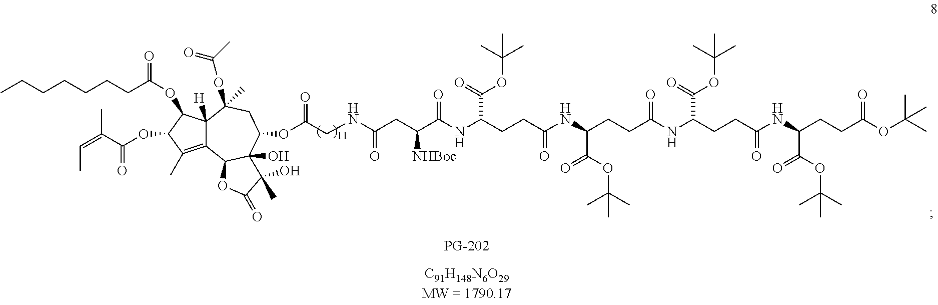

and

(e) converting the compound of Formula 8 into the compound of Formula 1.

Also provided herewith is more specific method for making the compound of Formula 1:

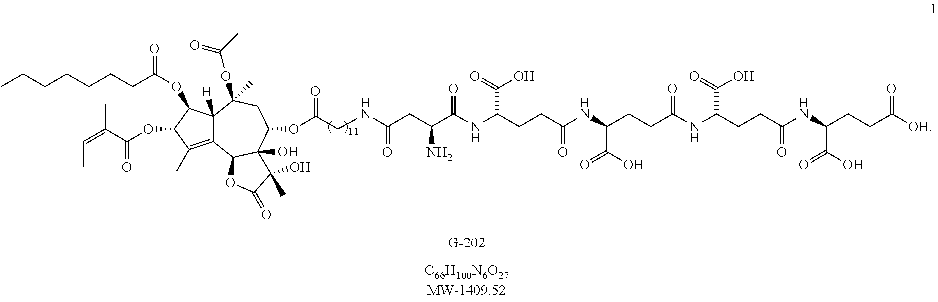

The method comprises:

(a) reacting the compound of Formula 2:

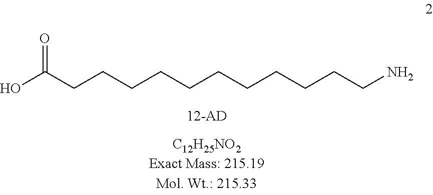 [SMILES: NCCCCCCCCCCCC(=O)O]

with benzyl alcohol (BnOH) in the presence of an acid catalyst to produce the compound of Formula 3:

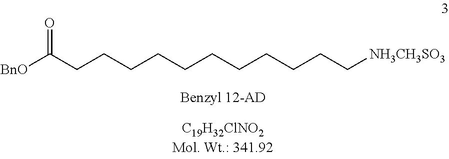

(b) reacting the compound of Formula 3 with the compound of Formula 4:

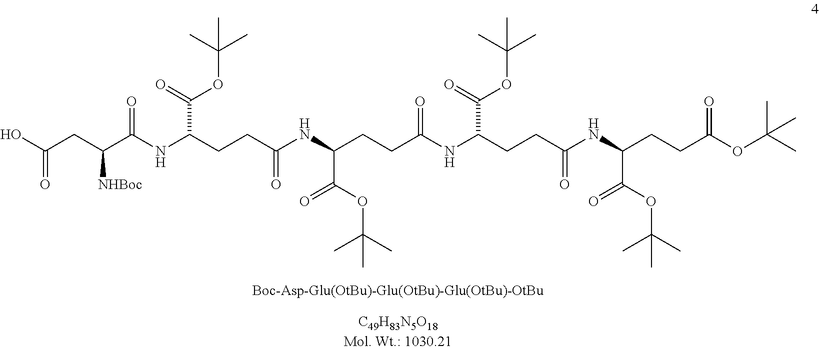 [SMILES: CC(C)(C)OC(=O)CC[C@H](NC(=O)CC[C@H](NC(=O)CC[C@H](NC(=O)CC[C@H](NC(=O)[C@H](CC(=O)O)NC(=O)OC(C)(C)C)C(=O)OC(C)(C)C)C(=O)OC(C)(C)C)C(=O)OC(C)(C)C)C(=O)OC(C)(C)C]

to produce the compound of Formula 5:

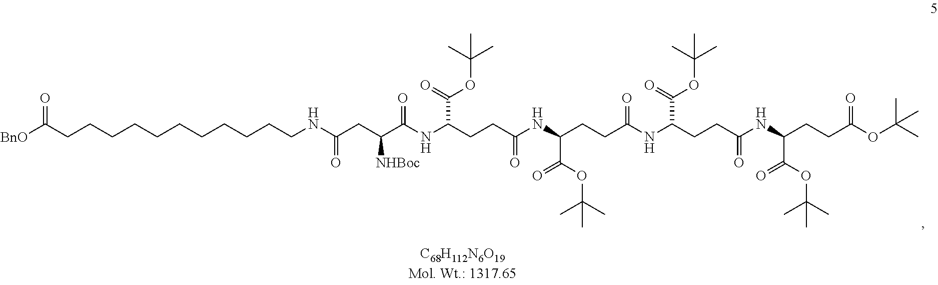 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)OCc1ccccc1)NC(=O)OC(C)(C)C)C(=O)OC(C)(C)C)C(=O)OC(C)(C)C)C(=O)OC(C)(C)C)C(=O)OC(C)(C)C]

(c) removing the benzyl group from the compound of Formula 5 to produce the compound of Formula 6:

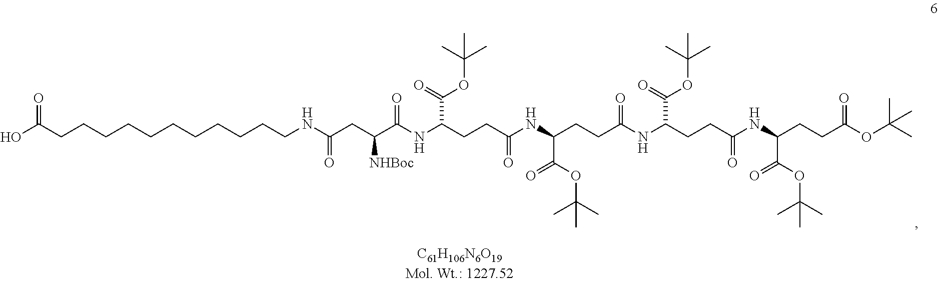 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]

(d) reacting the compound of Formula 6 with the compound of Formula 7:

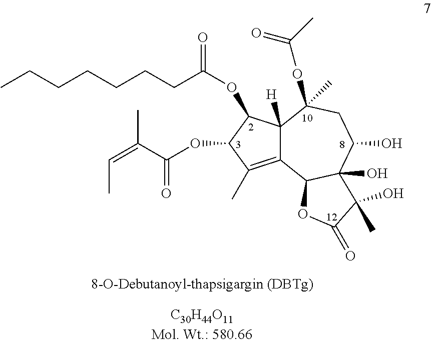 [SMILES: C/C=C(/C)C(=O)O[C@H]1C(C)=C2[C@H]([C@@H]1OC(=O)CCCCCCC)[C@@](C)(OC(C)=O)C[C@H](O)[C@@]1(O)[C@H]2OC(=O)[C@@]1(C)O]

to produce the compound of Formula 8:

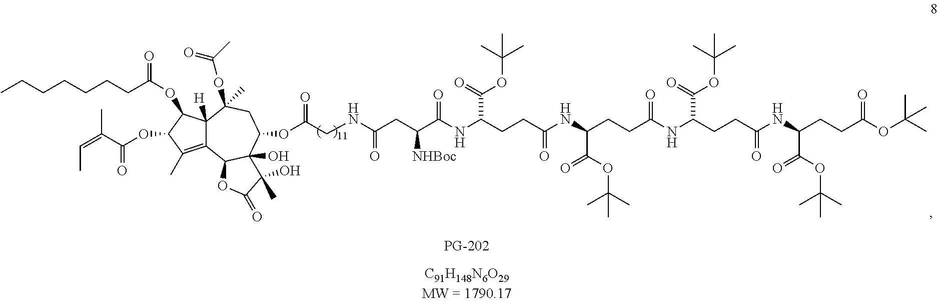

and

(e) converting the compound of Formula 8 into the compound of Formula 1.

Also provided is a compound having the formula of Formula 3, Formula 3a, Formula 5, Formula 5a or Formula 6.

Additionally, a method of making the compound of Formula 3a is provided. The method comprises reacting the compound of Formula 2 with X—OH in the presence of an acid catalyst.

In additional embodiments, a method of making the compound of Formula 5a is provided. The method comprises reacting the compound of Formula 3a with the compound of Formula 4.

Additionally provided is a method of making the compound of Formula 6. The method comprises removing the X group from the compound of Formula 5a.

Also provided is a compound having the formula XO—CO—(CH₂)ₙNH₂, wherein X is a substituent that forms an ester that can be cleaved from the compound of Formula 5a, and wherein n is an integer greater than 2.

Further provided is a method of making a compound having the formula XO—CO—(CH₂)ₙ—NH₂, as described immediately above. The method comprises reacting X—OH with HOOC—(CH₂)ₙ—NH₂ in the presence of an acid catalyst.

In further embodiments, a method of making a prodrug of a bioactive compound is provided. The method comprises:

(a) reacting a linker compound having the formula XO—CO—(CH₂)ₙ—NH₂ with a masking, targeting moiety (MTM) to form the compound XO-linker-MTM;
(b) removing the X group from the X-linker-MM compound to produce the compound OH-linker-MM; and
(c) reacting a bioactive compound with the compound OH-linker-MM to produce the prodrug of the bioactive compound.

### BRIEF DESCRIPTION OF THE DRAWINGS

FIG. 1 shows a procedure for producing DBTg (7) and Benzyl 12-AD (3).

FIG. 2 shows a procedure for producing the linker-peptide (6).

FIG. 3 shows a procedure for producing G-202 using 7 and 6.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

As used herein, the term “G-202” refers to 8-O-(12-aminododecanoyl)-8-O-debutanoyl-thapsigargin) aspartate-γ-glutamate-γ-glutamate-γ-glutamate-glutamate OH, having the chemical structure of Formula 1.

G-202 is a thapsigargin prodrug containing a cytotoxic analog of thapsigargin coupled to a masking and targeting peptide that inhibits its biologic activity until proteolytic cleavage at the tumor site. Thapsigargin itself is a natural product that is chemically modified to 8-O-debutanoyl-thapsigargin (DBTg). This thapsigargin analog (DBTg) is coupled the carboxylic acid of 12-aminododecanoic acid that has already been coupled to the beta carboxyl of Asp at the N-terminal end of the masking peptide Asp-γ-Glu-γ-Glu-γ-GluGlu to produce PG-202 which is subsequently deprotected to generate the prodrug (12ADT)-Asp-γ-Glu-γ-Glu-γ-GluGluOH (G-202).

The chemical name for G-202 is (8-O-(12-aminododecanoyl)-8-O-debutanoyl-thapsigargin) aspartate-γ-glutamate-γ-glutamate-γ-glutamate-glutamate OH. It is sometimes referred to in an abbreviated fashion: (12ADT)Asp-γ-Glu-γ-Glu-γ-Glu-GluOH, where 12ADT represents the thapsigargin derivative and Asp-γ-Glu-γ-Glu-γ-Glu-GluOH represents the PSMA-cleavable masking peptide. G-202 is a white solid with a molecular weight of 1409.52.

G-202 consists of a PSMA-selective 5 amino acid peptide substrate coupled to a highly cytotoxic analog of the natural product thapsigargin. See, e.g., Denmeade et al., 2003, and U.S. Pat. Nos. 7,767,648 and 7,468,354. Thapsigargin is isolated from the seeds of the plant Thapsia garganica, which grows as a weed throughout the Mediterranean basin. See, e.g., Rasmussen et al., 1978. Thapsigargin functions by potently inhibiting a critical intracellular protein, the sarcoplasmic/endoplasmic reticulum calcium ATPase (SERCA) pump whose normal function is to maintain intracellular calcium homeostasis in all cell types. Proper function of the SERCA pump is required for the viability of all cell types. Thus, thapsigargin inhibition of the SERCA pump results in the death of all cell types tested, both normal and malignant. See, e.g., Thastrup et al., 1990; Denmeade, 2005.

Based on the foregoing, G-202 and other thapsigargin prodrugs described herein were designed to target delivery of thapsigargin to a desired target site, e.g., tumors of various cancers, with a unique mechanism of action for selective activation of the prodrug by PSMA, for example PMSA produced by prostate cancer epithelial cells within sites of prostate cancer, and by tumor endothelial cells in other cancer cell types, for example, hepatocellular carcinoma or any other cancer that produces PSMA.

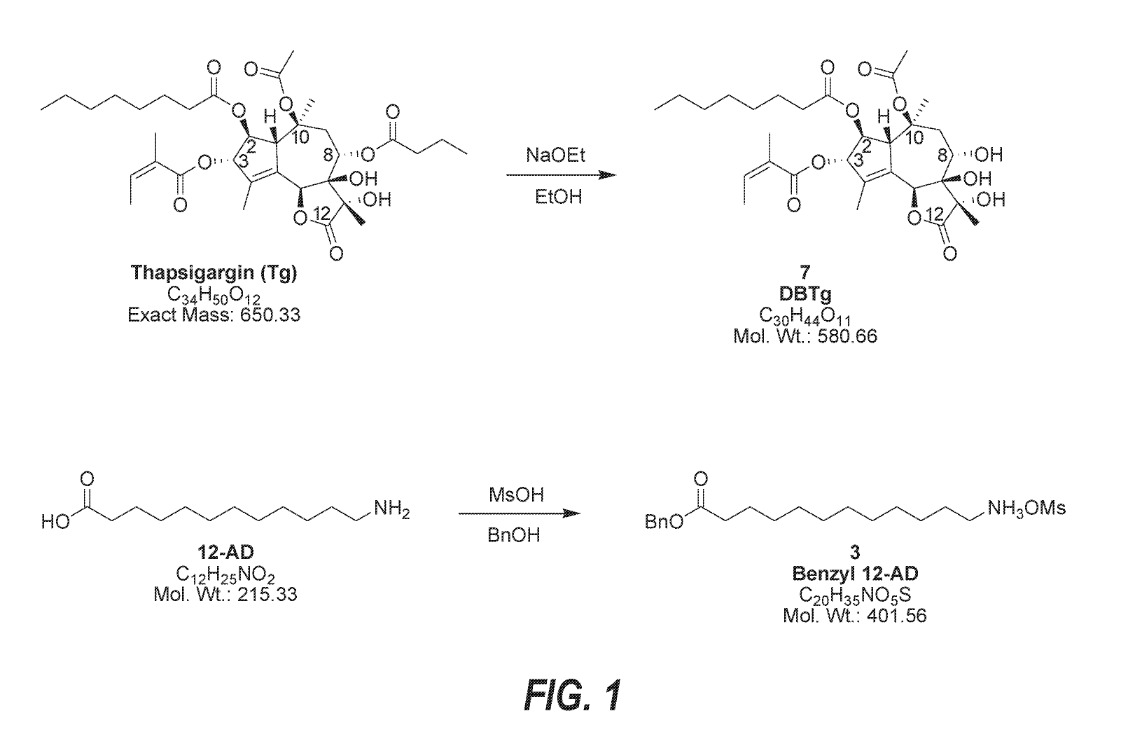

Without being bound by any particular theory, it is believed that PSMA is an extracellular carboxypeptidase that sequentially cleaves off acidic amino acids from the G-202 prodrug to eventually liberate a cytotoxic analog of thapsigargin. See, e.g., Pinto et al., 1996: Carter et al., 1996; Mhaka et al., 2004. This highly lipophilic analog, termed 12ADT-Asp, upon release from its water soluble peptide carrier, rapidly partitions into the surrounding cell membranes. See, e.g., Jakobsen et al., 2001. The analog then binds to the SERCA pump producing a sustained elevation in intracellular calcium which results in activation of apoptosis (see, e.g., FIG. 1; Denmeade et al., 2003; Singh et al., 2005). Because the 12ADT-Asp analog is released extracellularly into the tumor microenvironment, every cell does not need to produce PSMA to be killed by the prodrug activation. A substantial bystander effect is achieved by the release of the active drug into the tumor microenvironment.

Preclinical studies with G-202 have demonstrated that the prodrug is selectively activated by PSMA in vitro and is ˜60-fold more toxic to PSMA expressing vs. PSMA non-expressing tumor cells. PSMA shows significant growth inhibition against a panel of prostate, breast, renal, liver, and bladder cancers in vivo at doses that are minimally toxic to the host animal. See, e.g., Denmeade, S., et al., 2012.

U.S. Provisional Patent Application 61/791,909 describes a method for producing G-202 that involves production of 8-O-debutanoyl-thapsigargin (DBTg) from thapsigargin, and the linker Boc-aminododecanoate (AD-Boc), the linking of DBTg with AD-Boc to make Boc-12-aminododecanoate-thapsigargin (Boc-12ADT), the removal of the Boc group to form 12-ADT, and the linkage of 12-ADT with the peptide Boc-Asp-Glu(OtBu)-Glu(OtBu)-OtBu to form PG-202, which is then converted to G-202. The present invention provides modifications to that scheme to more easily produce G-202 with better yield.

Provided herewith is a method for making the compound of Formula 1:

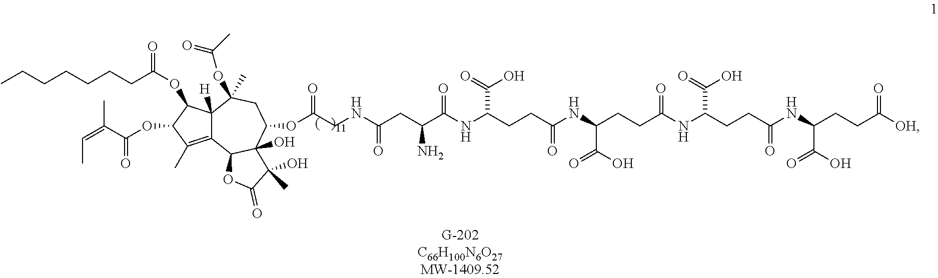

The method comprises

(a) reacting the compound of Formula 2:

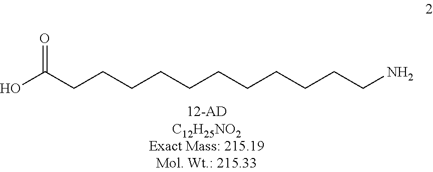 [SMILES: NCCCCCCCCCCCC(=O)O]

with X—OH in the presence of an acid catalyst to produce the compound of Formula 3a

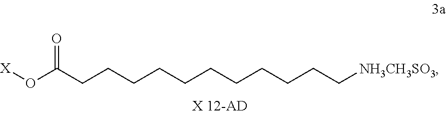

where X is a substituent that forms an ester that can be cleaved from the compound of Formula 5a:

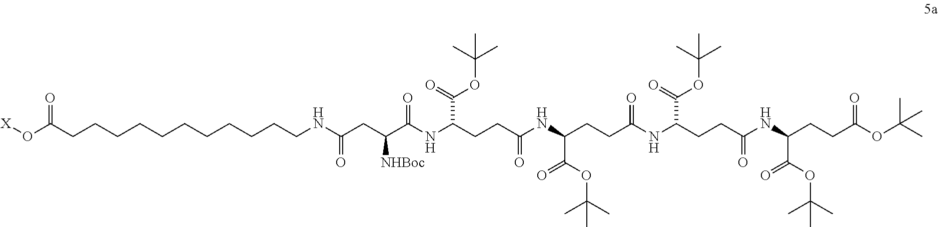

to produce the compound of Formula 6:

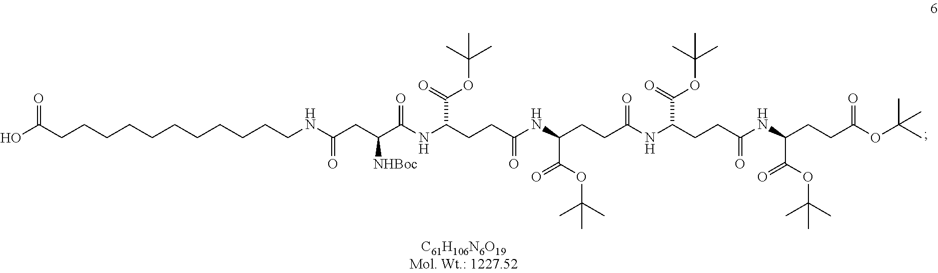 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]

(b) reacting the compound of Formula 3a with the compound of Formula 4:

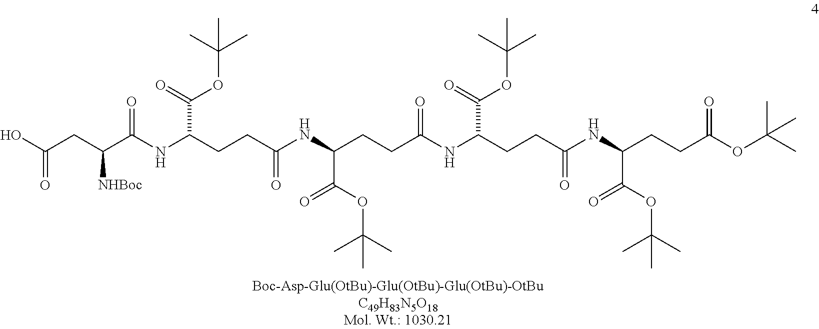 [SMILES: CC(C)(C)OC(=O)CC[C@H](NC(=O)CC[C@H](NC(=O)CC[C@H](NC(=O)CC[C@H](NC(=O)[C@H](CC(=O)O)NC(=O)OC(C)(C)C)C(=O)OC(C)(C)C)C(=O)OC(C)(C)C)C(=O)OC(C)(C)C)C(=O)OC(C)(C)C]

to produce the compound of Formula 5a;

(c) removing the X group from the compound of Formula 5a to produce the compound of Formula 6:

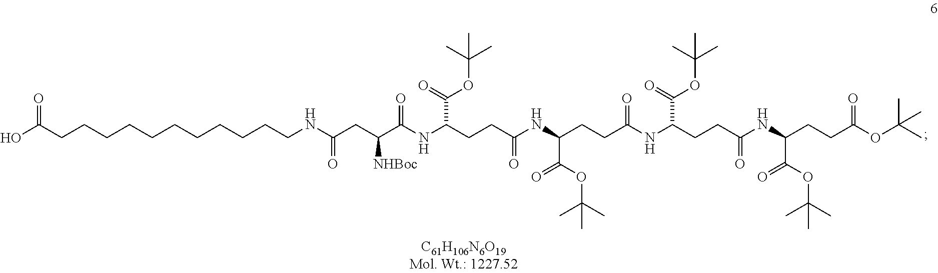 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]

(d) reacting the compound of Formula 6 with the compound of Formula 7:

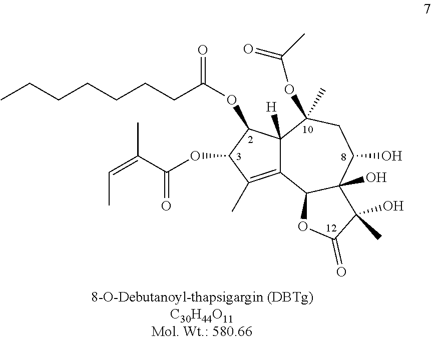 [SMILES: C/C=C(/C)C(=O)O[C@H]1C(C)=C2[C@H]([C@@H]1OC(=O)CCCCCCC)[C@@](C)(OC(C)=O)C[C@H](O)[C@@]1(O)[C@H]2OC(=O)[C@@]1(C)O]

to produce the compound of Formula 8:

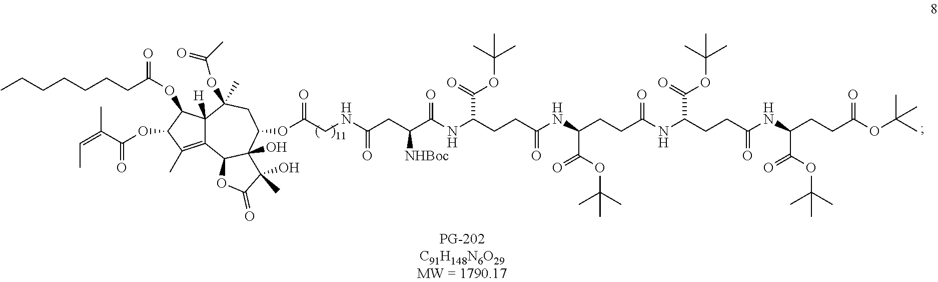

and

(e) converting the compound of Formula 8 into the compound of Formula 1.

In step (a) above, X—OH can be any alcohol that can make an ester that can be readily cleaved from the compound of Formula 5a. Such alcohols are readily recognizable to the skilled artisan without undue experimentation. For example, simple alkyl esters are not suitable, but substituted benzyl esters, allyl esters and aryl ester are generally effective. In some embodiments, X is benzyl alcohol.

In addition, numerous acid catalysts would work for the reaction of step (a) including, but not limited to, HCl, HBr, toluenesulfonic acid (TsOH), trifluoroacetic acid (TFA) and phosphoric acid. While step (a) above shows a methane sulfonic acid salt of benzyl 12-AD, the skilled artisan would understand that numerous other salts, as well as HCl, could be utilized. Those embodiments are not excluded from these methods.

The skilled artisan can determine, without undue experimentation, reactants that can be utilized in step (b) of the above method. In various embodiments, step (b) is performed using ethyl-(dimethylaminopropyl)carbodiimide (EDC), diisopropylethylamine (iPr2NEt), hydroxybenzotriazole (HOBt), and dimethylformamide (DMF).

Similarly, various methods would be understood by the skilled artisan for removing the X group in step (c). In some embodiments, step (c) is performed using a palladium on carbon (Pd/C) catalyst and triethylsilane. Other conditions effective at removing the X group includes palladium catalysis in the presence of alternate sources of hydrogen including, but not limited to, hydrogen gas, sodium formate, formic acid, or cyclohexene.

The reaction of step (d) can also be performed utilizing numerous conditions and methods that could be identified without undue experimentation. In some embodiments, step (d) is performed using 4-dimethylaminopyridine (4-DMAP) and diisopropylcarbodiimide (DIC) in dichloromethane. In other embodiments, alternate carbodiimide reagents (for example ethyldimethylaminopropylcarbodiimide (EDC)), mixed anhydride methods (for example pivalyl chloride/base) and phosphorous activating agents (for example propane phosphonic acid anhydride T3P) can be utilized, as is known in the art.

In various embodiments of these methods, step (e) is performed using triethylsilane and trifluoroacetic acid in dichloromethane. Alternate acid conditions such as hydrochloric acid in dioxane are also effective.

In some embodiments of these methods, the compound of Formula 7 is made by reacting the compound of Formula 9:

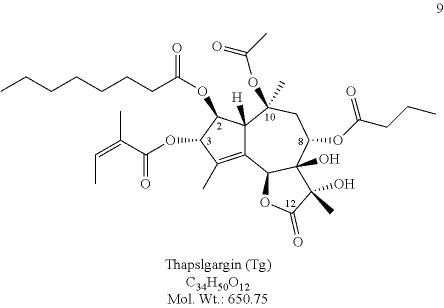 [SMILES: C/C=C(/C)C(=O)O[C@H]1C(C)=C2[C@H]([C@@H]1OC(=O)CCCCCCC)[C@@](C)(OC(C)=O)C[C@H](OC(=O)CCC)[C@@]1(O)[C@H]2OC(=O)[C@@]1(C)O]

with a base in an alcohol. Nonlimiting examples of useful base-alcohol combinations here are triethylamine and ethanol, sodium methoxide, and t-butoxide and alcohols and morpholine. In some embodiments, the base and alcohol are sodium ethoxide in ethanol. Conditions useful for executing this reaction with any alcohol-base combination can be determined without undue experimentation. Where the base and alcohol are sodium ethoxide in ethanol, a useful temperature for executing the reaction is a temperature of −15±5° C.

Once the compound of Formula 1 is synthesized by the above-described methods, that compound may be further purified by any means known in the art. In some embodiments, the compound of Formula 1 is purified using reverse-phase chromatography, e.g., as described in U.S. Provisional Patent Application 61/791,909 and PCT/US2014/029674.

Also provided herewith is more specific method for making the compound of Formula 1:

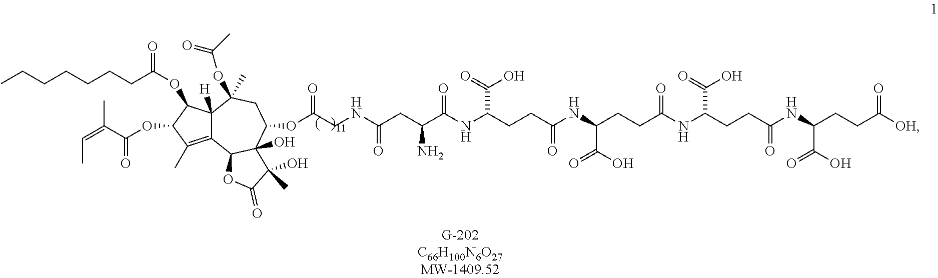

The method comprises

(a) reacting the compound of Formula 2:

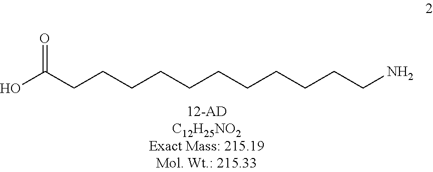 [SMILES: NCCCCCCCCCCCC(=O)O]

with benzyl alcohol (BnOH) in the presence of an acid catalyst to produce the compound of Formula 3 (the bottom reaction of FIG. 1):

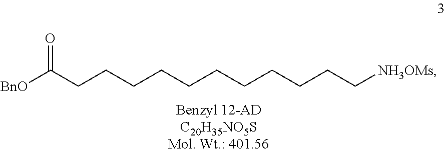

(b) reacting the compound of Formula 3 with the compound of Formula 4:

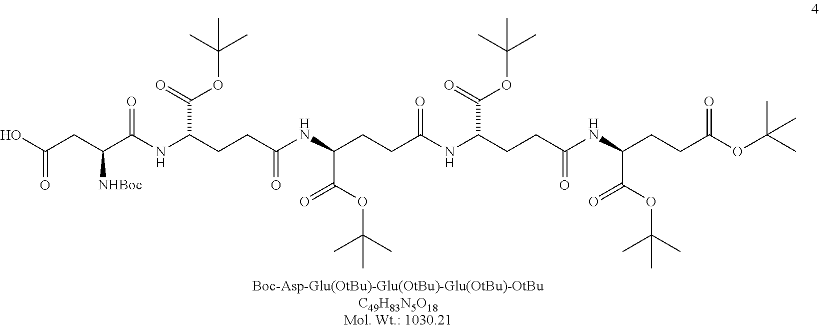 [SMILES: CC(C)(C)OC(=O)CC[C@H](NC(=O)CC[C@H](NC(=O)CC[C@H](NC(=O)CC[C@H](NC(=O)[C@H](CC(=O)O)NC(=O)OC(C)(C)C)C(=O)OC(C)(C)C)C(=O)OC(C)(C)C)C(=O)OC(C)(C)C)C(=O)OC(C)(C)C]

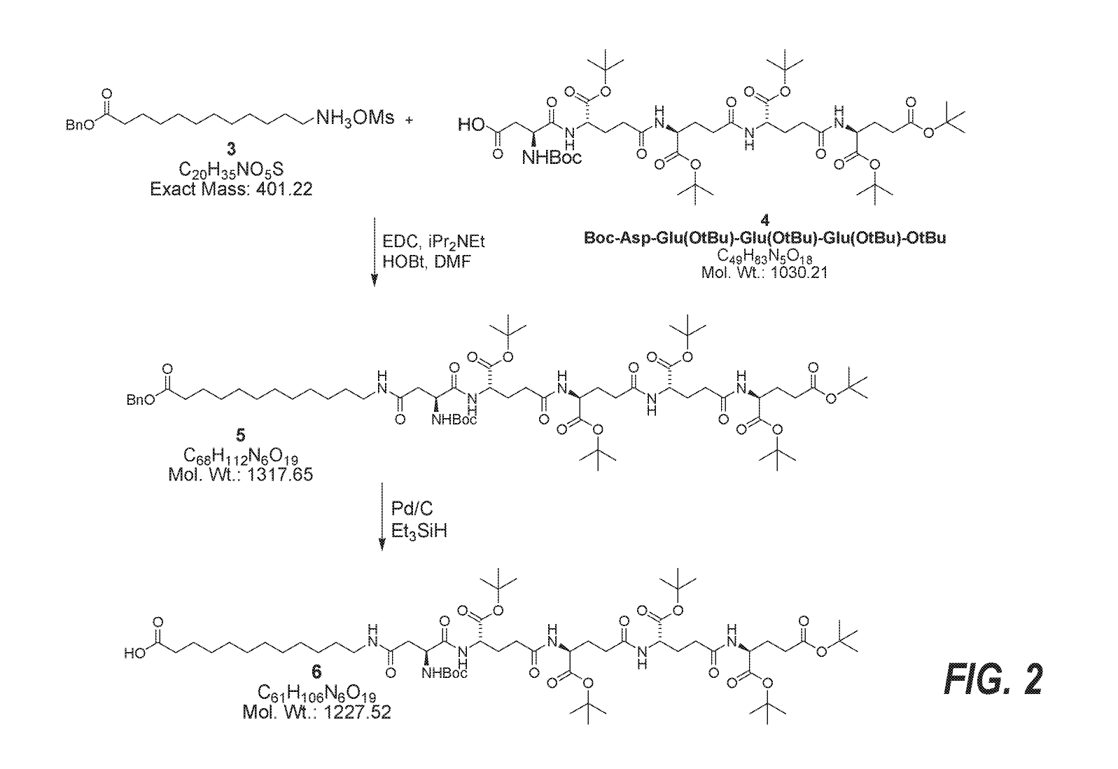

to produce the compound of Formula 5 (the upper reaction of FIG. 2):

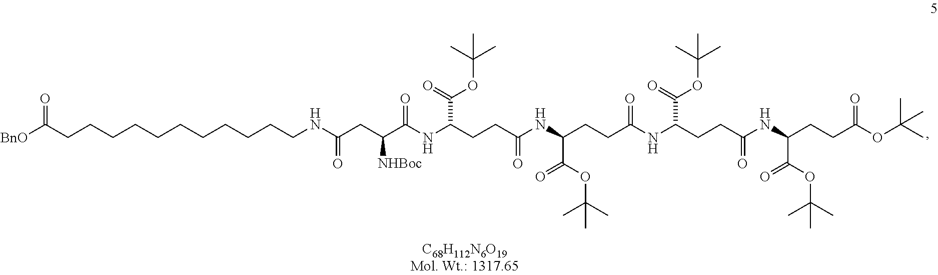 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)OCc1ccccc1)NC(=O)OC(C)(C)C)C(=O)OC(C)(C)C)C(=O)OC(C)(C)C)C(=O)OC(C)(C)C)C(=O)OC(C)(C)C]

(c) removing the benzyl group from the compound of Formula 5 to produce the compound of Formula 6 (the lower reaction of FIG. 1):

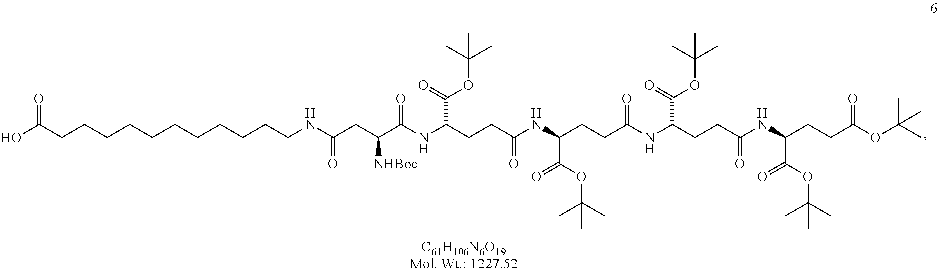 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]

(d) reacting the compound of Formula 6 with the compound of Formula 7:

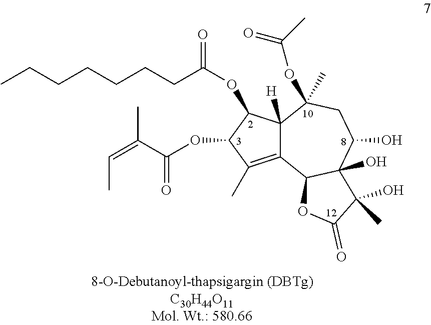 [SMILES: C/C=C(/C)C(=O)O[C@H]1C(C)=C2[C@H]([C@@H]1OC(=O)CCCCCCC)[C@@](C)(OC(C)=O)C[C@H](O)[C@@]1(O)[C@H]2OC(=O)[C@@]1(C)O]

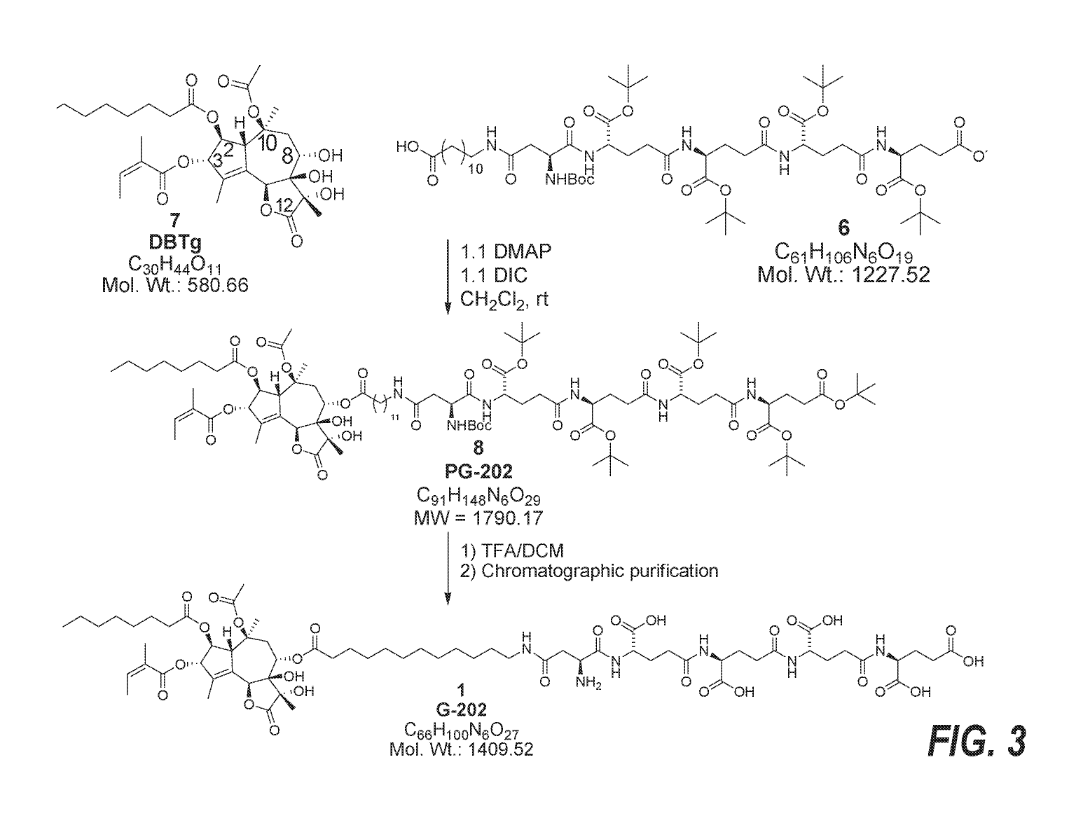

to produce the compound of Formula 8 (the upper reaction of FIG. 3):

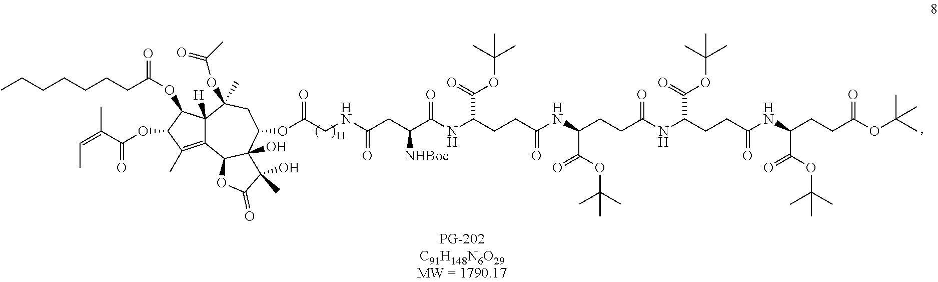

and

(e) converting the compound of Formula 8 into the compound of Formula 1 (the lower reaction of FIG. 3). Methods for this conversion step (e) are described in PCT/US2014/029674. The full discussion above with respect to the X moiety also specifically applies where X is benzyl in these embodiments.

The present invention also provides a compound having the formula of Formula 3a, Formula 5, Formula 5a or Formula 6. Those compounds are useful, for example, for making various prodrugs that are cleaved by peptidases such as PSMA.

Additionally provided is a method of making the compound of Formula 3a. The method comprises reacting the compound of Formula 2 with X—OH in the presence of an acid catalyst. As discussed above, X—OH can be any alcohol where X forms an ester that can be cleaved from the compound of Formula 5a. As previously discussed, non-limiting examples of such esters are substituted benzyl esters, allyl esters and aryl esters. In some embodiments, X—OH is benzyl alcohol.

Further provided is a method of making the compound of Formula 5a. The method comprises reacting the compound of Formula 3a with the compound of Formula 4.

In various embodiments, X a substituted benzyl, an allyl group or an aryl group. In additional embodiments, X is benzyl. In some embodiments, this method is performed using ethyl-(dimethylaminopropyl)carbodiimide (EDC), diisopropylethylamine (iPr2NEt), hydroxybenzotriazole (HOBt), and dimethylformamide (DMF).

The instant invention also provides a method of making the compound of Formula 6. The method comprises removing the X group from the compound of Formula 5a. In various embodiments, X a substituted benzyl, an allyl group or an aryl group. In additional embodiments, X is benzyl. In additional embodiments, this method is performed using a palladium on carbon (Pd/C) catalyst and triethylsilane.

Also provided is a compound having the formula XO—CO—(CH₂)ₙ—NH₂, where X is a substituent that forms an ester that can be cleaved from the compound of Formula 5a, and wherein n is an integer greater than 2, for example 3, 4, 5, 6, 7, 8, 9, 10, 11, 12, 13, 14, 15, 16, 17, 18, 19, 20 or any higher number. In some embodiments, X is substituted benzyl, an allyl group or an aryl group. In additional embodiments, X is benzyl. In further embodiments, n=11. In more specific embodiments, X is benzyl and n=11, as in the compound of Formula 3. Methods of making this compound are also provided. In some embodiments, the method comprises reacting X—OH (e.g., benzyl alcohol) with HOOC—(CH₂)ₙ—NH₂ in the presence of an acid catalyst.

The present invention also provides a method of making a prodrug of a bioactive compound. The method comprises
a) reacting the linker compound described above having the formula XO—CO—(CH₂)ₙ—NH₂ with a masking moiety (MM) to form the compound X-linker-MM;
b) removing the X group from the X-linker-MM compound to produce the compound OH-linker-MM; and
c) reacting a bioactive compound with the compound OH-linker-MM to produce the prodrug of the bioactive compound.

Similar to several of the methods and compounds described above, X is a substituent that forms an ester that can be cleaved from the linker, for example a substituted benzyl, an allyl group or an aryl group. In some embodiments, the X is benzyl; in other embodiments, n=11; in additional embodiments, X is benzyl and n=11.

This method is particularly useful for making a prodrug, for example when MM is a peptide and the bioactive compound is a cellular toxin, e.g., thapsigargin or another compound described in U.S. Pat. No. 7,468,354. In some embodiments, the prodrug is susceptible to cleavage by a peptidase, for example prostate specific membrane antigen (PSMA), to “unmask” the cellular toxin. In various embodiments of this method, the linker compound is the compound of Formula 3. In further embodiments, MM is the compound of Formula 4. In additional embodiments, the bioactive compound is modified before step (c).

Some advantages of this synthetic route to G-202 over the route described in U.S. Provisional Patent Application 61/791,909 are (a) avoidance of constructing and isolating the relatively unstable intermediate 12-ADT; (b) shorter and more efficient synthesis of the linker fragment; and (c) all intermediates are solids that can be isolated in high yield and purity.

Exemplary embodiments are described in the following example. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the example.

### Example 1. Synthesis of G-202

Preparation of 12-AD Benzyl Ester Mesylate Salt (3)

A N₂ flushed 3N 500 mL round bottom flask (RBF) was charged with 20 g (0.0929 mol, 1.0 eq.) of 12-aminododecanoic acid (12-AD-2) followed by 100 mL of benzyl alcohol and a thick suspension formed upon stirring. 6.63 mL (0.1022 mol, 1.1 eq.) of methane sulfonic acid (MSA) was added over ca. 10 minutes via addition funnel. An exothermic reaction brought the temperature from 22° C. to 34° C. The mixture was heated to ca. 50° C. for a total of 3 hours and cooled to room temperature. At ca. 30° C. the product began to precipitate from solution. The solution was stirred for ca. 10 minutes to let the product precipitate until a thick slurry formed. Acetone (200 mL) was added while stirring over ca. 20 minutes via addition funnel. The resulting slurry was stirred at room temperature for 30 minutes and further cooled to <5° C. for an additional hour and filtered to isolate the product. The product was washed in 2×100 mL cold acetone on the filter and transferred to a tared jar. The product was dried under vacuum overnight in the vacuum oven at 50° C. to give 35.14 g of crude product, a 94.2% recovery, 88.6% area HPLC and contained ca. 11% by area benzyl alcohol.

Recrystallization of Crude 12-AD Benzyl Ester Mesylate Salt (3)

A 1 L flask was charged with 33.0 g of the crude salt followed by 165 mL (5 mL/g) of ethanol. The resulting slurry was heated to ca. 40° C. and between 40-44° C. a clear colorless solution was formed. The heat was turned off to begin to cool the solution to room temperature. At ca. 35-38° C. a very thick slurry developed. Via addition funnel over ca. 20 minutes, 330 mL (10 mL/g) of acetone was added at 33 to 23° C. The slurry was stirred for ca. 30 minutes at room temperature and cooled to <5° C. with a dry ice water bath. After 1 hour at <5° C. the product was isolated by filtration. The product was washed on the funnel 3 times with 75 mL of cold acetone dried a short time on the filter and transferred to a tared jar. The product was dried in the vacuum oven at 50° C. under vacuum to give 28.33 g of product as a white crystalline solid 100% by area HPLC, a recovery of 85.8%, an overall yield of 80.8%.

Preparation of Benzyl Ester Coupled Peptide (5)

A nitrogen flushed 3N 250 mL RBF was charged with 12.0 g (0.0117 mol, 1.0 eq.) of G202 peptide (4), 4.91 g (0.0122 mol, 1.05 eq.) 12-AD Bn ester mesylate salt (3), 3.92 g (0.0256 mol, 2.2 eq.) HOBt, and 120 mL (10 mL/g) DMF. The stirrer was started and 5.1 mL (0.0291 mol, 2.5 eq.) of iPr2NEt (Hunigs base) was added with continued stirring for ca. 10 minutes until a yellow solution formed. 2.46 g (0.0128 mol, 1.1 eq.) EDC-HCl was added, and the solution was heated to ca. 50° C. for 2 hours. The reaction was complete by HPLC with 0.15% area peptide remaining. After a total of 2 hours 45 minutes the heat was turned off and the solution cooled to ca. 25° C. and transferred to a 1 L separatory funnel. The reaction was diluted with 400 mL of methyl tert-butyl ether (MTBE) and the organics were washed 3 times with 60 mL of 0.5M HCl, 2×60 mL of saturated NaHCO₃ and 1×60 mL of brine. The organic layer was dried over Na₂SO₄ and filtered. The drying salts were rinsed with MTBE and filtrate was concentrated to a gel like solid on a rotary evaporator. MTBE (200 mL) was added and stirred on a rotary evaporator at 40° C. for 15-20 minutes to form a solution. The solution was concentrated to a semi-solid and the product was dried for two days at ca. 40° C. in a vacuum oven to give 14.71 g of product 97.9% by area HPLC (G202BN3.M), a recovery of 95.8%.

Preparation of Debenzylated Coupled Peptide (6)

A nitrogen flushed 3N 250 mL RBF was charged with 12.0 g (0.00911 mol, 1.0 eq.) of Benzyl ester coupled peptide (5), 0.969 g (0.000273 mol, 0.03 eq.) palladium on carbon (5%, 60% wet) and 120 mL of isopropanol. The stirrer was started and the mixture stirred for ca. 15 minutes at room temperature. Triethylsilane 4.38 mL (0.0273 mol, 3.0 eq.) was added via addition funnel over 20 minutes at 19-25° C. there was gas evolution throughout the addition. One hour after the TES addition was complete, the reaction was checked for completion via HPLC and no starting material was detected. The reaction mixture was filtered through paper to remove the majority of the catalyst/carbon and then a second time through a 0.45 micro filter to remove the fines. The clear solution was concentrated on a rotary evaporator to a viscous clear oil. A 100 mL ACN distillation was performed to remove residual IPA. The product was dissolved in 82 mL ACN/6.8 mL H₂O at ca. 40° C. on a rotary evaporator with stirring and transferred to a 250 mL separatory funnel. The flask was rinsed forward to the separatory funnel with 26.2 mL ACN/5.2 mL H₂O (total: 108.2 mL ACN/12 mL H₂O). The aqueous layer was washed in 3×27 mL of heptane and the aqueous layer was concentrated to an oily/solid. ACN distilations (2×60 mL) were performed to remove residual water, followed by 2×60 mL of dichloromethane (DCM), to form a glassy foam/solid. The product was dried on a rotary evaporator under full vacuum at ca. 40° C. for ca. 45 minutes and broken up with a spatula. The product was further dried overnight in a vacuum oven at ca. 50° C. to give 11.37 g of product, 97.6% by area HPLC (G202BN3.M), a recovery of 101%.

Preparation of PG-202 (8)

A nitrogen flushed 3N RBF was charged with 12.0 g (0.0098 mol, 1.02 eq.) of the debenzylated coupled peptide (6), 5.56 g (0.0096 mol, 1.0 eq.) of DBTg (7), 1.4 g (0.0115 mol, 1.2 eq.) of 4-DMAP and 300 mL of DCM. The stirrer was started and the mixture stirred for ca. 5-10 minutes until a clear yellow solution formed. At room temperature 2.75 g (0.0144 mol, 1.5 eq.) of EDC-HCl was added all at once. There was no exothermic reaction, and the EDC-HCl went into solution quickly. The solution was stirred at room temperature overnight and checked for completion. There was ca. 0.2% of the reactants remaining. The reaction mixture was concentrated to a foam on a rotary evaporator to remove the DCM. The residue was dissolved in 180 mL MTBE/27 mL of 0.5M HCl at ca. 35° C. on a rotary evaporator and the two phase mixture was transferred to a reparatory funnel. The mixture was shaken vigorously and the layers were allowed to separate. The layers were separated the organics washed with 2×27 mL of 0.5M HCl, 1×27 mL of saturated NaHCO₃ and 1×27 mL of brine. The organics were dried over Na₂SO₄ and filtered; the drying salts were washed with MTBE. The solution was concentrated on a rotary evaporator to a foam/oil. The residue was dissolved in 120 mL MTBE at ca. 35° C. on a rotary evaporator and the solution was concentrated to a foam and the product placed under vacuum for ca. 30-40 minutes. The foam was broken up with a spatula and further dried overnight under vacuum in a vacuum oven at ca. 45° C. to give 16.62 g of product, 96.3% by area HPLC (G202BN3.M), a recovery of 95.7% (based on area % HPLC of starting material and product).

Preparation of G-202 (1)

A N₂ flushed 3N 250 mL RBF was charged with 10.0 g (0.00559 mol, 1.1 eq.) PG 202 (8) followed by 73 mL of DCM and the stirrer started. The mixture was stirred for ca. 5-10 minutes until a yellow solution formed. Triethylsilane (TES) 4.48 mL (0.0279 mol, 5 eq.) was added all at once and the solution was cooled to <5° C. Trifluoroacetic acid 39 mL was added via addition funnel over ca. 15 minutes at <5° C. and the solution stirred at <5° C. for 30 minutes before warming to room temperature. The solution was stirred at room temperature for ca. 10 hours (86.6% product by area % HPLC) and placed in the freezer overnight (88.8% product by area % HPLC at this point). The yellow solution was concentrated to remove DCM and some of the TFA. Two by 50 mL DCM distillations were done in order to remove more TFA. The residue was dissolved in 90 mL ACN/10 mL H₂O and the aqueous layer washed 2×10 mL of heptane. While stirring, 1 g of activated carbon was added to the aqueous layer and the dark mixture was stirred at room temperature for 30 minutes. The mixture was filtered through paper to remove the majority of the carbon and then through a 0.45 micron filter to remove the fines, resulting in a clear very light yellow solution. The solution was concentrated on a rotary evaporator to remove the ACN/H₂O; two additional 50 ml ACN distillations were performed to remove most of the water. Finally, 2×50 mL MTBE distillations were performed to give a product that was an off white foam/solid. The product was dried under vacuum on a rotary evaporator for ca. 30 minutes at ca. 40° C. at which point some of the material was a free flowing solid. The product was broken up with a spatula and the bulk was dried over two days at 45° C. in a vacuum oven to give 8.40 g of crude G202, 88.7% by area HPLC G202BN3.M) a recovery of 98.7%. The product was 70.7% by weight (T618) and 87.6% by area on this method.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference in their entirety and intended merely to summarize the assertions made by the authors. No admission is made that any reference constitutes prior art.

### REFERENCES

Carter et al., 1996, Proc. Natl. Acad. Sci., USA 93:749-753.
Denmeade et al., 2003, J. Natl. Cancer Inst. 9: 990-1000.
Denmeade, 2005, Cancer Biol. Ther. 4:14-22.
Denmeade et al., 2012, Sci. Transl. Med. 4:1-12.
Jakobsen et al., 2001, J. Med. Chem. 44:4696-4703.
Mhaka et al., 2004, Cancer Biol Ther. 3:551-558.
Pinto et al., 1996, Clin. Cancer Res. 2:1445-1451.
Rasmussen et al., 1978, Acta Pharm. Suec. 15:133-140.
Singh et al., 2005, J. Med. Chem. 48:3005-3014.
Thastrup et al., 1990, Proc. Natl. Acad. Sci. USA 87:2466-2470.
U.S. Pat. No. 7,767,648.
U.S. Pat. No. 7,468,354.
U.S. Provisional Patent Application 61/714,662.
U.S. Provisional Patent Application 61/693,273.
U.S. Provisional Patent Application 61/791,909.
PCT Application No. PCT/US2014/029674.

What is claimed is:

1. A method for making the compound of Formula 1: 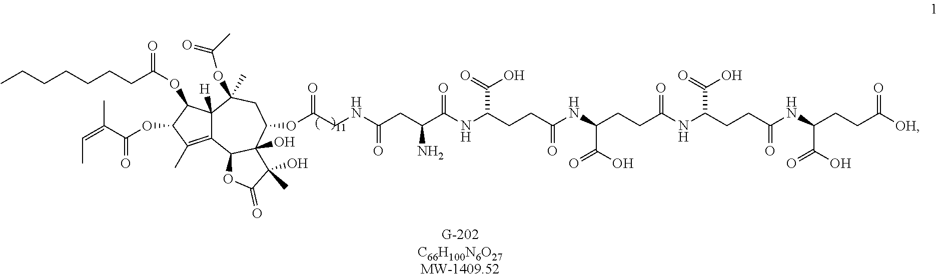
the method comprising
(a) reacting the compound of Formula 2: 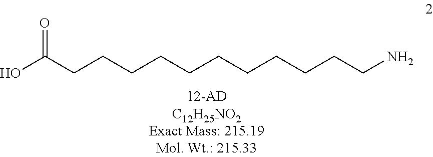 [SMILES: NCCCCCCCCCCCC(=O)O]
with X—OH in the presence of an acid catalyst to produce the compound of Formula 3a 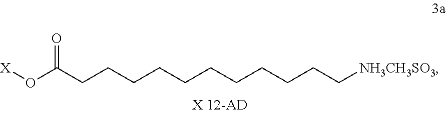
wherein X is a substituent that forms an ester that can be cleaved from the compound of Formula 5a 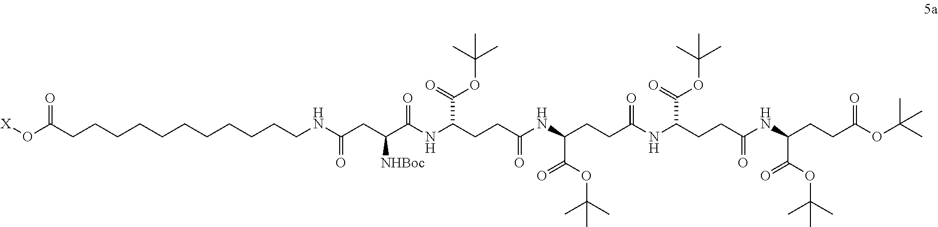
to produce the compound of Formula 6: 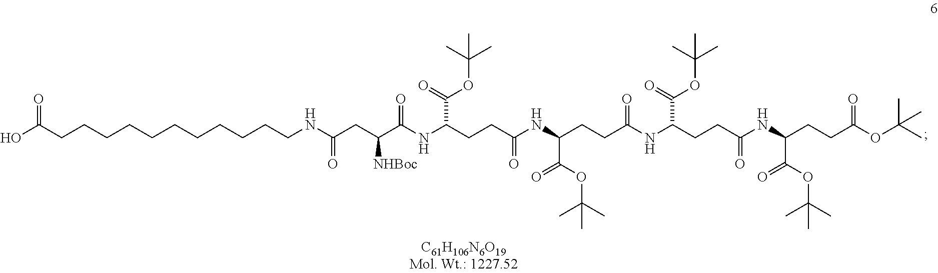 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]
(b) reacting the compound of Formula 3a with the compound of Formula 4: 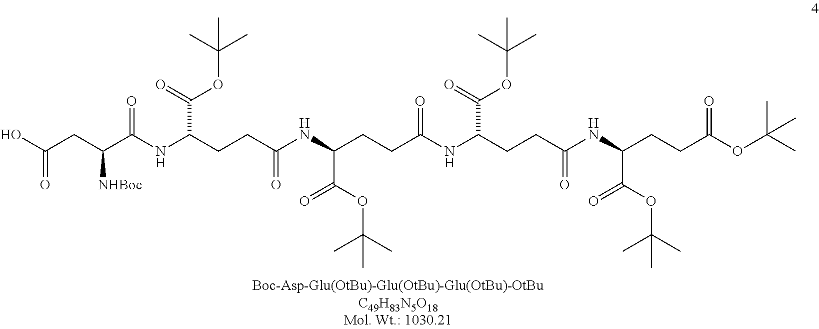 [SMILES: CC(C)(C)OC(=O)CC[C@H](NC(=O)CC[C@H](NC(=O)CC[C@H](NC(=O)CC[C@H](NC(=O)[C@H](CC(=O)O)NC(=O)OC(C)(C)C)C(=O)OC(C)(C)C)C(=O)OC(C)(C)C)C(=O)OC(C)(C)C)C(=O)OC(C)(C)C]
to produce the compound of Formula 5a;
(c) removing the X group from the compound of Formula 5a to produce the compound of Formula 6: 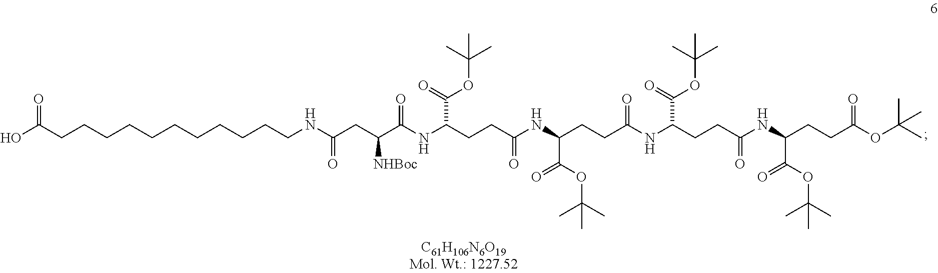 [SMILES: CC(C)(C)OC(=O)CC[C@H](NC(=O)CC[C@H](NC(=O)CC[C@H](NC(=O)CC[C@H](NC(=O)[C@H](CC(=O)NCCCCCCCCCCCC(=O)O)NC(=O)OC(C)(C)C)C(=O)OC(C)(C)C)C(=O)OC(C)(C)C)C(=O)OC(C)(C)C)C(=O)OC(C)(C)C]
(d) reacting the compound of Formula 6 with the compound of Formula 7: 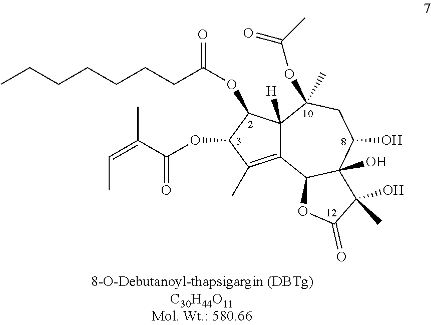 [SMILES: C/C=C(/C)C(=O)O[C@H]1C(C)=C2[C@H]([C@@H]1OC(=O)CCCCCCC)[C@@](C)(OC(C)=O)C[C@H](O)[C@@]1(O)[C@H]2OC(=O)[C@@]1(C)O]
to produce the compound of Formula 8: 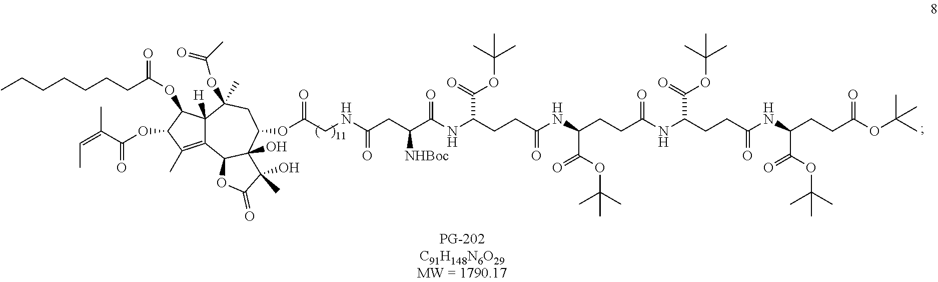
and
(e) converting the compound of Formula 8 into the compound of Formula 1.

2. The method of claim 1, wherein the compound of Formula 7 is made by reacting the compound of Formula 9: 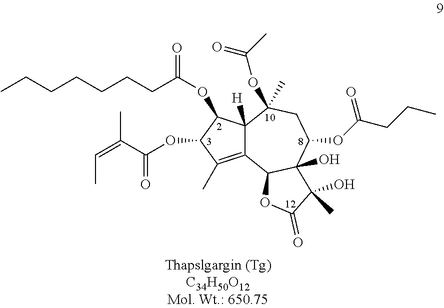 [SMILES: C/C=C(/C)C(=O)O[C@H]1C(C)=C2[C@H]([C@@H]1OC(=O)CCCCCCC)[C@@](C)(OC(C)=O)C[C@H](OC(=O)CCC)[C@@]1(O)[C@H]2OC(=O)[C@@]1(C)O]
with a base in an alcohol.

3. The method of claim 2, wherein the base in the alcohol are sodium ethoxide in ethanol.

4. The method of claim 1, wherein step (b) is performed using ethyl-(dimethylaminopropyl)carbodiimide (EDC), diisopropylethylamine (iPr2NEt), hydroxybenzotriazole (HOBt), and dimethylformamide (DMF).

5. The method of claim 1, wherein step (c) is performed using a palladium on carbon (Pd/C) catalyst and triethylsilane.

6. The method of claim 1, wherein step (d) is performed using 4-dimethylaminopyridine (4-DMAP) and diisopropylcarbodiimide (DIC) in dichloromethane.

7. The method of claim 1, wherein step (e) is performed using triethylsilane and trifluoroacetic acid in dichloromethane.

8-11. (canceled)

12. A compound having the formula of Formula 3, Formula 3a, Formula 5, Formula 5a or Formula 6.

13-20. (canceled)

21. A compound having the formula XO—CO—(CH₂)ₙNH₂, wherein X is a substituent that forms an ester that can be cleaved from the compound of Formula 5a, 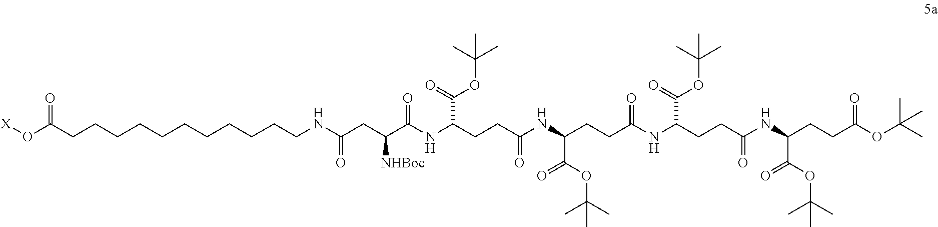
and wherein n is an integer greater than 2.

22. The compound of claim 21, wherein X is benzyl.

23. The compound of claim 21, wherein n=11.

24-26. (canceled)

27. A method of making a prodrug of a bioactive compound, the method comprising
(a) reacting the compound of Formula 3a with a masking moiety (MM) to form the compound X-linker-MM;
(b) removing an X group from the X-linker-MM compound to produce the compound OH-linker-MM; and
(c) reacting a bioactive compound with the compound OH-linker-MM to produce the prodrug of the bioactive compound.

28. The method of claim 27, wherein the MM is a peptide.

29. The method of claim 27, wherein the MM can be cleaved by prostate specific membrane antigen.

30. The method of claim 27, wherein X is benzyl.

31. The method of claim 27, wherein n=11.

32. The method of claim 27, wherein the linker compound is the compound of Formula 3.

33. (canceled)

34. The method of claim 27, wherein the bioactive compound is a thapsigargin.

35. The method of claim 27, wherein the bioactive compound is modified before step (c).

36. (canceled)

37. The method of claim 1, wherein X is benzyl and the compound of Formula 3a is Benzyl 12-AD having Formula 3: [chemical structure]
